(19) United States
(12) Patent Application Publication
(10) Pub. No.: US 2005/0129670 A1
(43) Pub. Date: Jun. 16, 2005

(54) Tumor cells modified to express B7-2 with increased immunogenicity and uses therefor

(75) Inventors: Gordon J. Freeman, Brookline, MA (US); Lee M. Nadler, Newton, MA (US); Gary S. Gray, Brookline, MA (US)

Correspondence Address: LAHIVE & COCKFIELD, LLP., 28 STATE STREET, BOSTON, MA, 02109, US

(73) Assignees: GENETICS INSTITUTE, LLC., Cambridge, MA (US); Dana-Farber Cancer Institute, Inc., Boston, MA (US)

(21) Appl. No.: 10/767,561

(22) Filed: Jan. 28, 2004

Related U.S. Application Data
(62) Division of application No. 09/206,132, filed on Dec. 7, 1998, now Pat. No. 6,723,705.
Application No. 09/206,132 is a division of application No. 08/456,104, filed on May 30, 1995, now Pat. No. 5,861,310.
Application No. 08/456,104 is a continuation-in-part of application No. 08/280,757, filed on Jul. 26, 1994, now Pat. No. 6,130,316.
Application No. 08/280,757 is a continuation-in-part of application No. 08/147,773, filed on Nov. 3, 1993, now abandoned.
Application No. 08/147,773 is a continuation-in-part of application No. 08/109,393, filed on Aug. 19, 1993, now abandoned.
Application No. 08/109,393 is a continuation-in-part of application No. 08/101,624, filed on Jul. 26, 1993, now Pat. No. 5,942,607.

(30) Foreign Application Priority Data
Jul. 26, 1994 (WO) PCT/US94/08423

(51) Int. Cl. A61K 48/00

(52) U.S. Cl. 424/93.21; 514/44

(57) ABSTRACT

Tumor cells modified to express one or more T cell costimulatory molecules are disclosed. Preferred costimulatory molecules are B7-2 and B7-3. The tumor cells of the invention can be modified by transfection with nucleic acid encoding B7-2 and/or B7-3, by using an agent which induces or increases expression of B7-2 and/or B7-3 on the tumor cell or by coupling B7-2 and/or B7-3 to the tumor cell. Tumor cells modified to express B7-2 and/or B7-3 can be further modified to express B7. Tumor cells further modified to express MHC class I and/or class II molecules or in which expression of an MHC associated protein, the invariant chain, is inhibited are also disclosed. The modified tumor cells of the invention can be used in methods for treating a patient with a tumor, preventing or inhibiting metastatic spread of a tumor or preventing or inhibiting recurrence of a tumor. A method for specifically inducing a CD4⁺ T cell response against a tumor and a method for treating a tumor by modification of tumor cells in vivo are disclosed.

### RELATED APPLICATIONS

This application is a divisional of U.S. Ser. No. 09/206,132, filed Dec. 7, 1998, entitled “Tumor Cells Modified to Express B7-2 and B7-3 with Increased Immunogenicity and Uses Therefor”; which is a divisional of U.S. Ser. No. 08/456,104, filed May 30, 1995, issued as U.S. Pat. No. 5,861,210; which is a Continuation-in-part of U.S. Ser. No. 08/280,757, filed on Jul. 26, 1994, issued as U.S. Pat. No. 6,130,316; which is a Continuation-in-part of U.S. Ser. No. 08/147,773 filed Nov. 3, 1993; which is a Continuation-in-part of U.S. Ser. No. 08/109,393 filed on Aug. 19, 1993; which is a Continuation-in-part of U.S. Ser. No. 08/101,624, filed Jul. 26, 1993, issued as U.S. Pat. No. 5,942,607. The contents of theses applications are specifically incorporated herein by reference.

### BACKGROUND OF THE INVENTION

Induction of a T lymphocyte response is a critical initial step in a host's immune response. Activation of T cells results in T cell proliferation, cytokine production by T cells and generation of T cell-mediated effector functions. T cell activation requires an antigen-specific signal, often called a primary activation signal, which results from stimulation of a clonally-distributed T cell receptor (hereafter TcR) present on the surface of the T cell. This antigen-specific signal is usually in the form of an antigenic peptide bound either to a major histocompatibility complex (hereafter MHC) class I protein or an MHC class II protein present on the surface of an antigen presenting cell (hereafter APC). CD4+ T cells recognize peptides associated with class II molecules. Class II molecules are found on a limited number of cell types, primarily B cells, monocytes/macrophages and dendritic cells, and, in most cases, present peptides derived from proteins taken up from the extracellular environment. In contrast, CD8+ T cells recognize peptides associated with class I molecules. Class I molecules are found on almost all cell types and, in most cases, present peptides derived from endogenously synthesized proteins. For a review see Germain, R., Nature 322, 687-691 (1986).

It has now been established that, in addition to an antigen-specific primary activation signal, T cells also require a second, non-antigen specific, signal to induce full T cell proliferation and/or cytokine production. This phenomenon has been termed co-stimulation. Mueller, D. L., et al., Annu. Rev. Immunol. 7, 445-480 (1989). Like the antigen-specific signal, the costimulatory signal is triggered by a molecule on the surface of the antigen presenting cell. A costimulatory molecule, the B lymphocyte antigen B7, has been identified on activated B cells and other APCs. Freeman, G. J., et al., J. Immunol. 139, 3260-3267 (1987); Freeman, G. J., et al., J. Immunol. 143, 2714-2722 (1989). Binding of B7 to a ligand on the surface of T cells provides costimulation to the T cell. Two structurally similar T cell-surface receptors for B7 have been identified, CD28 and CTLA-4. Aruffo, A. and Seed, B., Proc. Natl. Acad. Sci. USA 84, 8573-8577 (1987); Linsley, P. S., et al., J. Exp. Med. 173, 721-730, (1991); Brunet, J. F., et al., Nature 328, 267-270 (1987); Brunet, J. F., et al., Immunol Rev. 103, 21-36 (1988). CD28 is expressed constitutively on T cells and its expression is upregulated by activation of the T cell, such as by interaction of the TcR with an antigen-MHC complex. In contrast, CTLA4 is undetectable on resting T cells and its expression is induced by activation.

A series of experiments have shown a functional role for a T cell activation pathway stimulated through the CD28 receptor. Studies using blocking antibodies to B7 and CD28 have demonstrated that these antibodies can inhibit T cell activation, thereby demonstrating the need for stimulation via this pathway for T cell activation. Furthermore, suboptimal polyclonal stimulation of T cells by phorbol ester or anti-CD3 antibodies can be potentiated by crosslinking of CD28 with anti-CD28 antibodies. Engagement of the TcR by an MHC molecule/peptide complex in the absence of the costimulatory B7 signal can lead to T cell anergy rather than activation. Damle, N. K., et al., Proc. Natl. Acad. Sci. USA 78, 5096-5100 (1981); Lesslauer, W., et al., Eur. J. Immunol. 16, 1289-1295 (1986); Gimmi, C. D., et al., Proc. Natl. Acad. Sci. USA 88, 6575-6579 (1991); Linsley, P. S., et al., J. Exp. Med. 173; 721-730 (1991); Koulova, L., et al., J. Exp. Med. 173, 759-762 (1991); Razi-Wolf, Z., et al., Proc. Natl. Acad. Sci. USA 89, 4210-4214 (1992).

Malignant transformation of a cell is commonly associated with phenotypic changes in the cell. Such changes can include loss or gain of expression of some proteins or alterations in the level of expression of certain proteins. It has been hypothesized that in some situations the immune system may be capable of recognizing a tumor as foreign and, as such, could mount an immune response against the tumor. Kripke, M., Adv. Cancer Res. 34, 69-75 (1981). This hypothesis is based in part on the existence of phenotypic differences between a tumor cell and a normal cell, which is supported by the identification of tumor associated antigens (hereafter TAAs). Schreiber, H., et al. Ann. Rev. Immunol. 6, 465-483 (1988). TAAs are thought to distinguish a transformed cell from its normal counterpart. Three genes encoding TAAs expressed in melanoma cells, MAGE-1, MAGE-2 and MAGE-3, have recently been cloned. van der Bruggen, P., et al. Science 254, 1643-1647 (1991). That tumor cells under certain circumstances can be recognized as foreign is also supported by the existence of T cells which can recognize and respond to tumor associated antigens presented by MHC molecules. Such TAA-specific T lymphocytes have been demonstrated to be present in the immune repertoire and are capable of recognizing and stimulating an immune response against tumor cells when properly stimulated in vitro. Rosenberg, S. A., et al. Science 233, 1318-1321 (1986); Rosenberg, S. A. and Lotze, M. T. Ann. Rev. Immunol. 4, 681-709 (1986).

However, in practice, tumors in vivo have generally not been found to be very immunogenic and appear to be capable of evading immune response. This may result from an inability of tumor cells to induce T cell-mediated immune responses. Ostrand-Rosenberg, S., et al., J. Immunol. 144, 4068-4071 (1990); Fearon, E. R., et al., Cell 60, 397-403 (1990). A method for increasing the immunogenicity of a tumor cell in vivo would be therapeutically beneficial.

### SUMMARY OF THE INVENTION

Although most tumor cells are thought to express TAAs which distinguish tumor cells from normal cells and T cells which recognize TAA peptides have been identified in the immune repertoire, an anti-tumor T cell response may not be induced by a tumor cell due to a lack of costimulation necessary to activate the T cells. It is known that many tumors are derived from cells which do not normally function as antigen-presenting cells, and, thus, may not trigger necessary signals for T cell activation. In particular, tumor cells may be incapable of triggering a costimulatory signal in a T cell which is required for activation of the T cell. This invention is based, at least in part, on the discovery that tumor cells modified to express a costimulatory molecule, and therefore capable of triggering a costimulatory signal, can induce an anti-tumor T cell-mediated immune response in vivo. This T cell-mediated immune response is effective not only against the modified tumor cells but, more importantly, against the unmodified tumor cells from which they were derived. Thus, the effector phase of the anti-tumor response induced by the modified tumor cells of the invention is not dependent upon expression of a costimulatory molecule on the tumor cells.

Accordingly, the invention pertains to methods of inducing or enhancing T lymphocyte-mediated anti-tumor immunity in a subject by use of a modified tumor cell having increased immunogenicity. In one aspect of the invention, a tumor cell is modified to express one or more T cell costimulatory molecules on its surface. Preferred costimulatory molecules are novel B lymphocyte antigens, B7-2 and B7-3. Prior to modification, the tumor cell may lack the ability to express B7-2 and/or B7-3, may be capable of expressing B7-2 and/or B7-3 but fail to do so, or may express insufficient amounts of B7-2 and/or B7-3 to activate T cells. Therefore, a tumor cell can be modified by providing B7-2 and/or B7-3 to the tumor cell surface, by inducing the expression of B7-2 and/or B7-3 on the tumor cell or by increasing the level of expression of B7-2 and/or B7-3 on the tumor cell. In one embodiment, the tumor cell is modified by transfecting the cell with at least one nucleic acid encoding B7-2 and/or B7-3 in a form suitable for expression of the molecule(s) on the cell surface. Alternatively, the tumor cell is contacted with an agent which induces or increases expression of B7-2 and/or B7-3 on the cell surface. In yet another embodiment, the tumor cell is modified by chemically coupling B7-2 and/or B7-3 to the tumor cell surface. A tumor cell modified to express B7-2 and/or B7-3 can be further modified to express the T cell costimulatory molecule B7.

Even when provided with the ability to trigger a costimulatory signal in T cells, modified tumor cells may still be incapable of inducing anti-tumor T cell-mediated immune responses due to a failure to sufficiently trigger an antigen-specific primary activation signal. This can result from insufficient expression of MHC class I or class II molecules on the tumor cell surface. Accordingly, this invention encompasses modified tumor cells which provide both a T cell costimulatory signal and an antigen-specific primary activation signal, via an antigen-MHC complex, to T cells. Prior to modification, a tumor cell may lack the ability to express one or more MHC molecules, may be capable of expressing one or more MHC molecules but fail to do so, may express only certain types of MHC molecules (e.g., class I but not class II), or may express insufficient amounts of MHC molecules to activate T cells. Thus, in one embodiment, a tumor cell is modified by providing one or more MHC molecules to the tumor cell surface, by inducing the expression of one or more MHC molecules on the tumor cell surface or by increasing the level of expression of one or more MHC molecules on the tumor cell surface. Tumor cells expressing B7-2 and/or B7-3 are further modified, for example, by transfection with a nucleic acid encoding one or more MHC molecules in a form suitable for expression of the MHC molecule(s) on the tumor cell surface. Alternatively, such tumor cells are modified by contact with an agent which induces or increases expression of one or more MHC molecules on the cell.

In a particularly preferred embodiment, tumor cells modified to express B7-2 and/or B7-3 are further modified to express one or more MHC class II molecules. To provide an MHC class II molecule, at least one nucleic acid encoding an MHC class II a chain protein and an MHC class II β chain protein are introduced into the tumor cell such that expression of these proteins is directed to the surface of the cell. In yet another embodiment, tumor cells modified to express B7-2 and/or B7-3 are further modified to express one or more MHC class I molecules. To provide an MHC class I molecule, at least one nucleic acid encoding an MHC class I α chain protein and a β-2 microglobulin protein are introduced such that expression of these proteins is directed to the surface of the tumor cell. Alternatively, a tumor cell modified to express B7-2 and/or B7-3 can be further modified by contact with an agent which induces or increases the expression of MHC molecules (class I and/or class II) on the cell surface.

In certain situations, modified tumor cells of the invention may fail to activate T cells because of insufficient association of TAA-derived peptides with MHC molecules, resulting in a lack of an antigen-specific primary activation signal in T cells. Accordingly, the invention further pertains to a tumor cell modified to trigger a costimulatory signal in T cells and in which association of TAA peptides with MHC class II molecules is promoted in order to induce an antigen-specific signal in T cells. This aspect of the invention is based, at least in part, on the ability of an MHC class II associated protein, the invariant chain, to prevent association of endogenously derived peptides (which would include a number of TAA peptides) with MHC class II molecules intracellularly. Thus, in one embodiment, a tumor cell modified to express B7-2 and/or B7-3 is further modified to promote association of TAA peptides with MHC class II molecules by inhibiting the expression of the invariant chain in the tumor cell. The tumor cell selected to be so modified can be one which naturally expresses both MHC class II molecules and the invariant chain or can be one-which expresses the invariant chain and which has been modified to express MHC class II molecules. Preferably, expression of the invariant chain is inhibited in a tumor cell by introducing into the tumor cell a nucleic acid which is antisense to a coding or regulatory region of the invariant chain gene. Alternatively, expression of the invariant chain in a tumor cell is prevented by an agent which inhibits expression of the invariant chain gene or which inhibits expression or activity of the invariant chain protein.

The modified tumor cells of the invention can be used in methods for inducing an anti-tumor T lymphocyte response in a subject effective against both modified and unmodified tumor cells. For example, tumor cells can be obtained, modified as described herein to trigger a costimulatory signal in T lymphocytes, and administered to the subject to elicit a T cell-mediated immune response. The modified tumor cells of the invention can also be administered to prevent or inhibit metastatic spread of a tumor or to prevent or inhibit recurrence of a tumor following therapeutic treatment.

This invention also provides methods for treating a subject with a tumor by modifying tumor cells in vivo to be capable of triggering a costimulatory signal in T cells, and, if necessary, also an antigen-specific signal.

The tumor cells of the current invention modified to express B7-2 and/or B7-3 and one or more MHC class II molecules can be used in a method for specifically inducing an anti-tumor response by CD4+ T lymphocytes in a subject with a tumor by administering the modified tumor cells to the subject. Alternatively, a CD4+ T cell response can be induced by modifying tumor cells in vivo to express a B7-2 and/or B7-3 and one or more MHC class II molecules.

The invention also pertains to a composition of modified tumor cells suitable for pharmaceutical administration. This composition comprises an amount of tumor cells and a physiologically acceptable carrier.

### BRIEF DESCRIPTION OF THE DRAWINGS

FIG. 1 shows graphs depicting the cell surface expression of B7 and the MHC class II molecule I-A^{k} on wild-type and transfected tumor cells as determined by immunofluourescent staining of the cells.

FIG. 2 is a graphic representation of tumor cell growth (as measured by tumor size) in mice following transplantation of J558 plasmacytoma cells or J558 plasmacytoma cells transfected to express B7-1 (J558-B7.1) or B7-2 (J558-B7.2).

### DETAILED DESCRIPTION OF THE INVENTION

The induction of a T cell response requires that at least two signals be delivered by ligands on a stimulator cell to the T cell through cell surface receptors on the T cell. A primary activation signal is delivered to the T cell through the antigen-specific TcR. Physiologically, this signal is triggered by an antigen-MHC molecule complex on the stimulator cell, although it can also be triggered by other means such as phorbol ester treatment or crosslinking of the TcR complex with antibodies, e.g. with anti-CD3. To induce T cell activation, a second signal, called a costimulatory signal, is required by stimulation of the T cell through another cell surface molecule, such as CD28 or CTLA4. Thus, the minimal molecules on a stimulator cell required for T cell activation are an MHC molecule associated with a peptide antigen, to trigger a primary activation signal in a T cell, and a costimulatory molecule to trigger a costimulatory signal in the T cell. Engagement of the antigen-specific TcR in the absence of triggering of a costimulatory signal can prevent activation of the T cell and, in addition, can induce a state of unresponsiveness or anergy in the T cells.

In addition to the previously characterized B lymphocyte activation antigen B7, human B lymphocytes express other novel molecules which costimulate T cell activation. These costimulatory molecules include antigens on the surface of B lymphocytes, professional antigen presenting cells (e.g., monocytes, dendritic cells, Langerhan cells) and other cells which present antigen to immune cells (e.g., keratinocytes, endothelial cells, astrocytes, fibroblasts, oligodendrocytes) and which bind either CTLA4, CD28, both CTLA4 and CD28 or other known or as yet undefined receptors on immune cells. Novel B lymphocyte antigens which provide cotimulation to activated T cells to thereby induce T cell proliferation and/or cytokine secretion include the B7-2 (human and mouse) and the B7-3 antigens described herein.

The B lymphocyte antigen B7-2 is expressed by human B cells at about 24 hours following stimulation with either anti-immunoglobulin or anti-MHC class II monoclonal antibody. The B7-2 antigen induces detectable IL-2 secretion and T cell proliferation. At about 48 to 72 hours post activation, human B cells express both B7 and a third CTLA4 counter-receptor, B7-3, identified by a monoclonal antibody BB-1, which also binds B7 (Yokochi, T., et al. (1982) J. Immunol. 128, 823-827). The B7-3 antigen is also expressed on B7 negative activated B cells and can costimulate T cell proliferation without detectable IL-2 production, indicating that the B7 and B7-3 molecules are distinct. B7-3 is expressed on a wide variety of cells including activated B cells, activated monocytes, dendritic cells, Langerhan cells and keratinocytes. At 72 hours post B cell activation, the expression of B7 and B7-3 begins to decline. The presence of these costimulatory molecules on the surface of activated B lymphocytes indicates that T cell costimulation is regulated, in part, by the temporal expression of these molecules following B cell activation.

The ability of a tumor cell to evade an immune response and fail to stimulate a T lymphocyte response against the cell may result from the inability of the cell to properly activate T cells. This invention provides modified tumor cells which trigger a costimulatory signal in T cells and, thus; activate an anti-tumor T lymphocyte response. Tumor cells are modified to be capable of triggering a costimulatory signal by providing B7-2 and/or B7-3 to the tumors. Tumors cells may be further modified by providing B7. Additionally, in certain embodiments, tumor cells are modified to trigger both a primary, antigen-specific activation signal and a costimulatory signal in T cells.

The modified tumor cells of the invention display increased immunogenicity and can be used to induce or enhance a T cell-mediated immune response against a tumor. Since the effector phase of the T cell-mediated immune response is not dependent upon expression of a costimulatory molecule by tumor cells, the T cell-mediated immune response generated by administration of a modified tumor cell of the invention is effective against not only the modified tumor cells but also the unmodified tumor cells from which they were derived.

I. Ex Vivo Modification of a Tumor Cell to Express a Costimulatory Molecule

The inability of a tumor cell to trigger a costimulatory signal in T cells may be due to a lack of expression of a costimulatory molecule, failure to express a costimulatory molecule even though the tumor cell is capable of expressing such a molecule, insufficient expression of a costimulatory molecule on the tumor cell surface or lack of expression of an appropriate costimulatory molecule (e.g. expression of B7 but not B7-2 and/or B7-3). Thus, according to one aspect of the invention, a tumor cell is modified to express B7-2 and/or B7-3 by transfection of the tumor cell with a nucleic acid encoding B7-2 and/or B7-3 in a form suitable for expression of B7-2 and/or B7-3 on the tumor cell surface. Alternatively, the tumor cell is modified by contact with an agent which induces or increases expression of B7-2 and/or B7-3 on the tumor cell surface. In yet another embodiment, B7-2 and/or B7-3 is coupled to the surface of the tumor cell to produce a modified tumor cell.

The ability of a molecule, such as B7-2 or B7-3, to provide a costimulatory signal to T cells can be determined, for example, by contacting T cells which have received a primary activation signal with the molecule to be tested and determining the presence of T cell proliferation and/or cytokine secretion. T cell can be suboptimally stimulated with a primary activation signal, for instance by contact with immobilized anti-CD3 antibodies or a phorbol ester. Following this stimulation, the T cells are exposed to cells expressing B7-2 and/or B7-3 on their surface and the proliferation of the T cells and/or secretion of cytokines, such as IL-2, by the T cells is determined. Proliferation and/or cytokine secretion will be increased by triggering of a costimulatory signal in the T cells. T cell proliferation can be measured, for example, by a standard ³H-thymidine uptake assay. Cytokine secretion can be measured, for example, by a standard IL-2 assay. See for example Linsley, P. S., et al., J. Exp. Med. 173, 721-730 (1991), Gimmi, C. D., et al., Proc. Natl. Acad. Sci. USA 88: 6575-6579 (1991), Freeman, G. J., et al., J. Exp. Med. 174, 625-631, (1991).

Fragments, mutants or variants of B7-2 and/or B7-3 that retain the ability to interact with T cells, trigger a costimulatory signal and activate T cell responses, as evidenced by proliferation and/or cytokine production by T cells that have received a primary activation signal, are considered within the scope of the invention. A “fragment” of B7-2 and/or B7-3 is defined as a portion of B7-2 and/or B7-3 which retains costimulatory activity. For example, a fragment of B7-2 and/or B7-3 may have fewer amino acid residues than the entire protein. A “mutant” is defined as B7-2 and/or B7-3 having a structural change which may enhance, diminish, not affect, but not eliminate the costimulatory activity of the molecule. For example, a mutant of B7-2 and/or B7-3 may have a change in one or more amino acid residues of the protein. A “variant” is defined as B7-2 and/or B7-3 having a modification which does not affect the costimulatory activity of the molecule. For example, a variant of B7-2 and/or B7-3 may have altered glycosylation or may be a chimeric protein of the costimulatory molecule and another protein.

A. Transfection of a Tumor Cell with a Nucleic Acid Encoding a Costimulatory Molecule

Tumor cells can be modified ex vivo to express B7-2 and/or B7-3 by transfection of isolated tumor cells with a nucleic acid encoding B7-2 and/or B7-3 in a form suitable for expression of the molecule on the surface of the tumor cell. The terms “transfection” or “transfected with” refers to the introduction of exogenous nucleic acid into a mammalian cell and encompass a variety of techniques useful for introduction of nucleic acids into mammalian cells including electroporation, calcium-phosphate precipitation, DEAE-dextran treatment, lipofection, microinjection and infection with viral vectors. Suitable methods for transfecting mammalian cells can be found in Sambrook et al. (Molecular Cloning: A Laboratory Manual, 2nd Edition, Cold Spring Harbor Laboratory press (1989)) and other laboratory textbooks. The nucleic acid to be introduced may be, for example, DNA encompassing the gene(s) encoding B7-2 and/or B7-3, sense strand RNA encoding B7-2 and/or B7-3 or a recombinant expression vector containing a cDNA encoding B7-2 and/or B7-3. The nucleotide sequence of a cDNA encoding human B7-2 is shown in SEQ ID NO: 1, and the amino acid sequence of a human B7-2 protein is shown in SEQ ID NO:2. The nucleotide sequence of a cDNA encoding mouse B7-2 is shown in SEQ ID NO: 3, and the amino acid sequence of a mouse B7-2 protein is shown in SEQ ID NO:4.

The nucleic acid is “in a form suitable for expression of B7-2” or “in a form suitable for expression of B7-3” in which the nucleic acid contains all of the coding and regulatory sequences required for transcription and translation of a gene, which may include promoters, enhancers and polyadenylation signals, and sequences necessary for transport of the molecule to the surface of the tumor cell, including N-terminal signal sequences. When the nucleic acid is a cDNA in a recombinant expression vector, the regulatory functions responsible for transcription and/or translation of the cDNA are often provided by viral sequences. Examples of commonly used viral promoters include those derived from polyoma, Adenovirus 2, cytomegalovirus and Simian Virus 40, and retroviral LTRs. Regulatory sequences linked to the cDNA can be selected to provide constitutive or inducible transcription, by, for example, use of an inducible promoter, such as the metallothienin promoter or a glucocorticoid-responsive promoter. Expression of B7-2 or B7-3 on the surface of the tumor cell can be accomplished, for example, by including the native trans-membrane coding sequence of the molecule in the nucleic acid sequence, or by including signals which lead to modification of the protein, such as a C-terminal inositol-phosphate linkage, that allows for association of the molecule with the outer surface of the cell membrane.

A preferred approach for introducing nucleic acid encoding B7-2 and/or B7-3 into tumor cells is by use of a viral vector containing nucleic acid, e.g. a cDNA, encoding B7-2 and/or B7-3. Examples of viral vectors which can be used include retroviral vectors (Eglitis, M. A., et al., Science 230, 1395-1398 (1985); Danos, O. and Mulligan, R., Proc. Natl. Acad. Sci. USA 85, 6460-6464 (1988): Markowitz, D. et al. J. Virol. 62, 1120-1124 (1988)), adenoviral vectors (Rosenfeld, M. A., et al., Cell 68, 143-155 (1992)) and adeno-associated viral vectors (Tratschin, J. D., et al., Mol. Cell. Biol. 5, 3251-3260 (1985)). Infection of tumor cells with a viral vector has the advantage that a large proportion of cells will receive nucleic acid, thereby obviating a need for selection of cells which have received nucleic acid, and molecules encoded within the viral vector, e.g. by a cDNA contained in the viral vector, are expressed efficiently in cells which have taken up viral vector nucleic acid.

Alternatively, B7-2 and/or B7-3 can be expressed on a tumor cell using a plasmid expression vector which contains nucleic acid, e.g. a cDNA, encoding B7-2 and/or B7-3. Suitable plasmid expression vectors include CDM8 (Seed, B., Nature 329, 840 (1987)) and pMT2PC (Kaufman, et al., EMBO J. 6, 187-195 (1987)). Since only a small fraction of cells (about 1 out of 10⁵) typically integrate transfected plasmid DNA into their genomes, it is advantageous to transfect a nucleic acid encoding a selectable marker into the tumor cell along with the nucleic acid(s) of interest. Preferred selectable markers include those which confer resistance to drugs such as G418, hygromycin and methotrexate. Selectable markers may be introduced on the same plasmid as the gene(s) of interest or may be introduced on a separate plasmid. Following selection of transfected tumor cells using the appropriate selectable marker(s), expression of the costimulatory molecule on the surface of the tumor cell can be confirmed by immunofluorescent staining of the cells. For example, cells may be stained with a fluorescently labeled monoclonal antibody reactive against the costimulatory molecule or with a fluorescently labeled soluble receptor which binds the costimulatory molecule. Expression of the B7-3 costimulatory molecule can be determined using a monoclonal antibody, BB1, which recognizes B7-3. Yokochi, T., et al. J. Immunol. 128, 823-827 (1982). Alternatively, a labeled soluble CD28 or CTLA4 protein or fusion protein (e.g. CTLA4Ig) which binds to B7-2 and B7-3 can be used to detect expression of B7-2 and/or B7-3.

When transfection of tumor cells leads to modification of a large proportion of the tumor cells and efficient expression of B7-2 and/or B7-3 on the surface of tumor cells, e.g. when using a viral expression vector, tumor cells may be used without further isolation or subcloning. Alternatively, a homogenous population of transfected tumor cells can be prepared by isolating a single transfected tumor cell by limiting dilution cloning followed by expansion of the single tumor cell into a clonal population of cells by standard techniques.

B. Induction or Increased Expression of a Costimulatory Molecule on a Tumor Cell Surface

A tumor cell can be modified to trigger a costimulatory signal in T cells by inducing or increasing the level of expression of B7-2 and/or B7-3 on a tumor cell which is capable of expressing B7-2 and/or B7-3 but fails to do so or which expresses insufficient amounts of B7-2 and/or B7-3 to activate T cells. An agent which stimulates expression of B7-2 and/or B7-3 can be used in order to induce or increase expression of B7-2 and/or B7-3 on the tumor cell surface. For example, tumor cells can be contacted with the agent in vitro in a culture medium. The agent which stimulates expression of B7-2 and/or B7-3 may act, for instance, by increasing transcription of B7-2 and/or B7-3 gene, by increasing translation of B7-2 and/or B7-3 mRNA or by increasing stability or transport of B7-2 and/or B7-3 to the cell surface. For example, it is known that expression of B7 can be upregulated in a cell by a second messenger pathway involving cAMP. Nabavi, N., et al. Nature 360, 266-268 (1992). B7-2 and B7-3 may likewise be inducible by cAMP. Thus, a tumor cell can be contacted with an agent, which increases intracellular cAMP levels or which mimics cAMP, such as a cAMP analogue, e.g. dibutyryl cAMP, to stimulate expression of B7-2 and/or B7-3 on the tumor cell surface. It is also known that expression of B7 can be induced on normal resting B cells by crosslinking cell-surface MHC class II molecules on the B cells with an antibody against the MHC class II molecules. Kuolova, L., et al., J. Exp. Med. 173, 759-762 (1991). Similarly, B7-2 and B7-3 can be induced on resting B cells by crosslinking cell-surface MHC class II molecules on the B cells. Accordingly, a tumor cell which expresses MHC class II molecules on its surface can be treated with anti-MHC class II antibodies to induce or increase B7-2 and or B7-3 expression on the tumor cell surface.

Another agent which can be used to induce or increase expression of B7-2 and/or B7-3 on a tumor cell surface is a nucleic acid encoding a transcription factor which upregulates transcription of the gene encoding the costimulatory molecule. This nucleic acid can be transfected into the tumor cell to cause increased transcription of the costimulatory molecule gene, resulting in increased cell-surface levels of the costimulatory molecule.

C. Coupling of a Costimulatory Molecule to the Surface of a Tumor Cell

In another embodiment, a tumor cell is modified to be capable of triggering a costimulatory signal in T cells by coupling B7-2 and/or B7-3 to the surface of the tumor cell. For example, B7-2 and/or B7-3 molecules can be obtained using standard recombinant DNA technology and expression systems which allow for production and isolation of the costimulatory molecule(s). Alternatively, B7-2 and/or B7-3 can be isolated from cells which express the costimulatory molecule(s) using standard protein purification techniques. For example, B7-3 protein can be isolated from activated B cells by immunoprecipitation with an anti-B7-3 antibody such as the BB1 monoclonal antibody. The isolated costimulatory molecule is then coupled to the tumor cell. The terms “coupled” or “coupling” refer to a chemical, enzymatic or other means (e.g., antibody) by which B7-2 and/or B7-3 is linked to a tumor cell such that the costimulatory molecule is present on the surface of the tumor cell and is capable of triggering a costimulatory signal in T cells. For example, B7-2 and/or B7-3 can be chemically crosslinked to the tumor cell surface using commercially available crosslinking reagents (Pierce, Rockford Ill.). Another approach to coupling B7-2 and/or B7-3 to a tumor cell is to use a bispecific antibody which binds both the costimulatory molecule and a cell-surface molecule on the tumor cell. Fragments, mutants or variants of B7-2 and/or B7-3 which retain the ability to trigger a costimulatory signal in T cells when coupled to the surface of a tumor cell can also be used.

D. Modification of Tumor Cells to Express Multiple Costimulatory Molecules

Another aspect of the invention is a tumor cell modified to express multiple costimulatory molecules. The temporal expression of costimulatory molecules on activated B cells is different for B7, B7-2 and B7-3. For example, B7-2 is expressed early following B cell activation, whereas B7-3 is expressed later. The different costimulatory molecules may thus serve distinct functions during the course of an immune response. An effective T cell response may require that the T cell receive costimulatory signals from multiple costimulatory molecules. Accordingly, the invention encompasses a tumor cell which is modified to express more than one costimulatory molecule. For example, a tumor cell can be modified to express both B7-2 and B7-3. Alternatively, a tumor cell modified to express B7-2 can be further modified to express B7. Similarly, a tumor cell modified to express B7-3 can be further modified to express B7. A tumor cell can also be modified to express B7, B7-2 and B7-3.

Before modification, a tumor cell may not express any costimulatory molecules, or may express certain costimulatory molecules but not others. As described herein, tumor cells can be modified by transfecting the tumor cell with nucleic acid encoding a costimulatory molecule(s), by inducing the expression of a costimulatory molecule(s) or by coupling a costimulatory molecule(s) to the tumor cell. For example, a tumor cell transfected with nucleic acid encoding B7-2 can be further transfected with nucleic acid encoding B7. The cDNA sequence and deduced amino acid sequence of human and mouse B7 is shown in SEQ ID NO:5 and 6 and SEQ ID NO:7 and 8, respectively. Alternatively, more than one type of modification can be used. For example, a tumor cell transfected with a nucleic acid encoding B7-2 can be stimulated with an agent which induces expression of B7.

II. Additional Modification of a Tumor Cell to Express MHC Molecules

Another aspect of this invention features modified tumor cells which express a costimulatory molecule and which express one or more MHC molecules on their surface to trigger both a costimulatory signal and a primary, antigen-specific, signal in T cells. Before modification, tumor cells may be unable to express MHC molecules, may fail to express MHC molecules although they are capable of expressing such molecules, or may express insufficient amounts of MHC molecules on the tumor cell surface to cause T cell activation. Tumor cells can be modified to express either MHC class I or MHC class II molecules, or both. One approach to modifying tumor cells to express MHC molecules is to transfect the tumor cell with one or more nucleic acids encoding one or more MHC molecules. Alternatively, an agent which induces or increases expression of one or more MHC molecules on tumor cells can be used to modify tumor cells. Inducing or increasing expression of MHC class II molecules on a tumor cell can be particularly beneficial for activating CD4+ T cells against the tumor since the ability of MHC class II⁺ tumor cells to directly present tumor peptides to CD4⁺ T cells bypasses the need for professional MHC class II⁺ APCs. This can improve tumor immunogenicity because soluble tumor antigen (in the form of tumor cell debris or secreted protein) may not be available for uptake by professional MHC class II⁺ APCs.

One embodiment of the invention is a modified tumor cell which expresses B7-2 and/or B7-3 and one or more MHC class II molecules on their cell surface. MHC class II molecules are cell-surface α/β heterodimers which structurally contain a cleft into which antigenic peptides bind and which function to present bound peptides to the antigen-specific TcR. Multiple, different MHC class II proteins are expressed on professional APCs and different MHC class II proteins bind different antigenic peptides. Expression of multiple MHC class II molecules, therefore, increases the spectrum of antigenic peptides that can be presented by an APC or by a modified tumor cell. The α and β chains of MHC class II molecules are encoded by different genes. For instance, the human MHC class II protein HLA-DR is encoded by the HLA-DRα and HLA-DRβ genes. Additionally, many polymorphic alleles of MHC class II genes exist in human and other species. T cells of a particular individual respond to stimulation by antigenic peptides in conjunction with self MHC molecules, a phenomenon termed MHC restriction. In addition, certain T cells can also respond to stimulation by polymorphic alleles of MHC molecules found on the cells of other individuals, a phenomenon termed allogenicity. For a review of MHC class II structure and function, see Germain and Margulies, Ann. Rev. Immunol. 11: 403-450, 1993.

Another embodiment of the invention is a modified tumor cell which expresses B7-2 and/or B7-3 and one or more MHC class I molecules on the cell surface. Similar to MHC class II genes, there are multiple MHC class I genes and many polymorphic alleles of these genes are found in human and other species. Like MHC class II proteins, class I proteins bind peptide fragments of antigens for presentation to T cells. A functional cell-surface class I molecule is composed of an MHC class I α chain protein associated with a β2-microglobulin protein.

A. Transfection of a Tumor Cell with Nucleic Acid Encoding MHC Molecules

Tumor cells can be modified ex vivo to express one or more MHC class II molecules by transfection of isolated tumor cells with one or more nucleic acids encoding one or more MHC class II α chains and one or more MHC class II β chains in a form suitable for expression of the MHC class II molecules(s) on the surface of the tumor cell. Both an α and a β chain protein must be present in the tumor cell to form a surface heterodimer and neither chain will be expressed on the cell surface alone. The nucleic acid sequences of many murine and human class II genes are known. For examples see Hood, L., et al. Ann. Rev. Immunol. 1, 529-568 (1983) and Auffray, C. and Strominger, J. L. Advances in Human Genetics 15, 197-247 (1987). Preferably, the introduced MHC class II molecule is a self MHC class II molecule. Alternatively, the MHC class II molecule could be a foreign, allogeneic, MHC class II molecule. A particular foreign MHC class II molecule to be introduced into tumor cells can be selected by its ability to induce T cells from a tumor-bearing subject to proliferate and/or secrete cytokines when stimulated by cells expressing the foreign MHC class II molecule (i.e. by its ability to induce an allogeneic response). The tumor cells to be transfected may not express MHC class II molecules on their surface prior to transfection or may express amounts insufficient to stimulate a T cell response. Alternatively, tumor cells which express MHC class II molecules prior to transfection can be further transfected with additional, different MHC class II genes or with other polymorphic alleles of MHC class II genes to increase the spectrum of antigenic fragments that the tumor cells can present to T cells.

Fragments, mutants or variants of MHC class II molecules that retain the ability to bind peptide antigens and activate T cell responses, as evidenced by proliferation and/or lymphokine production by T cells, are considered within the scope of the invention. A preferred variant is an MHC class II molecule in which the cytoplasmic domain of either one or both of the α and β chains is truncated. It is known that truncation of the cytoplasmic domains allows peptide binding by and cell surface expression of MHC class II molecules but prevents the induction of endogenous B7 expression, which is triggered by an intracellular signal generated by the cytoplasmic domains of the MHC class II protein chains upon crosslinking of cell surface MHC class II molecules. Kuolova. L., et al., J. Exp. Med. 173, 759-762 (1991); Nabavi, N., et al. Nature 360, 266-268 (1992). Expression of B7-2 and B7-3 is also induced by crosslinking surface MHC class II molecules, and thus truncation of MHC class II molecules may also prevent induction of B7-2 and/or B7-3. In tumor cells transfected to constitutively express B7-2 and/or B7-3, it may be desirable to inhibit the expression of endogenous costimulatory molecules, for instance to restrain potential downregulatory feedback mechanisms. Transfection of a tumor cell with a nucleic acid(s) encoding a cytoplasmic domain-truncated form of MHC class II α and β chain proteins would inhibit endogenous B7 expression and possibly also endogenous B7-2 and B7-3 expression. Such variants can be produced by, for example, introducing a stop codon in the MHC class II chain gene(s) after the nucleotides encoding the transmembrane spanning region. The cytoplasmic domain of either the α chain or the β chain protein can be truncated, or, for more complete inhibition of B7 (and possibly B7-2 and/or B7-3) induction, both the α and β chains can be truncated. See e.g. Griffith et al., Proc. Natl. Acad. Sci. USA 85: 4847-4852, (1988), Nabavi et al., J. Immunol. 142: 1444-1447, (1989).

Tumor cells can be modified to express an MHC class I molecule by transfection with a nucleic acid encoding an MHC class I α chain protein. For examples of nucleic acids see Hood, L., et al. Ann. Rev. Immunol. 1, 529-568 (1983) and Auffray, C. and Strominger, J. L., Advances in Human Genetics 15, 197-247 (1987). Optionally, if the tumor cell does not express β-2 microglobulin, it can also be transfected with a nucleic acid encoding the β-2 microglobulin protein. For examples of nucleic acids see Gussow, D., et al., J. Immunol. 139, 3132-3138 (1987) and Parnes, J. R., et al., Proc. Natl. Acad. Sci. USA 78, 2253-2257 (1981). As for MHC class II molecules, increasing the number of different MHC class I genes or polymorphic alleles of MHC class I genes expressed in a tumor cell can increase the spectrum of antigenic fragments that the tumor cells can present to T cells.

When a tumor cell is transfected with nucleic acid which encodes more than one molecule, for example a B7-2 and/or B7-3 molecule(s), an MHC class II α chain protein and an MHC class II β chain protein, the transfections can be performed simultaneously or sequentially. If the transfections are performed simultaneously, the molecules can be introduced on the same nucleic acid, so long as the encoded sequences do not exceed a carrying capacity for a particular vector used. Alternatively, the molecules can be encoded by separate nucleic acids. If the transfections are conducted sequentially and tumor cells are selected using a selectable marker, one selectable marker can be used in conjunction with the first introduced nucleic acid while a different selectable marker can be used in conjunction with the next introduced nucleic acid.

The expression of MHC molecules (class I or class II) on the cell surface of a tumor cell can be determined, for example, by immunoflourescence of tumor cells using fluorescently labeled monoclonal antibodies directed against different MHC molecules. Monoclonal antibodies which recognize either non-polymorphic regions of a particular MHC molecule (non-allele specific) or polymorphic regions of a particular MHC molecule (allele-specific) can be used and are known to those skilled in the art.

B. Induction or Increased Expression of MHC Molecules on a Tumor Cell

Another approach to modifying a tumor cell ex vivo to express MHC molecules on the surface of a tumor cell is to use an agent which stimulates expression of MHC molecules in order to induce or increase expression of MHC molecules on the tumor cell surface. For example, tumor cells can be contacted with the agent in vitro in a culture medium. An agent which stimulates expression of MHC molecules may act, for instance, by increasing transcription of MHC class I and/or class II genes, by increasing translation of MHC class I and/or class II mRNAs or by increasing stability or transport of MHC class I and/or class II proteins to the cell surface. A number of agents have been shown to increase the level of cell-surface expression of MHC class II molecules. See for example Cockfield, S. M. et al., J. Immunol. 144, 2967-2974 (1990); Noelle, R. J. et al. J. Immunol. 137, 1718-1723 (1986); Mond, J. J., et al., J. Immunol. 127, 881-888 (1981); Willman, C. L., et al. J. Exp. Med., 170, 1559-1567 (1989); Celada, A. and Maki, R. J. Immunol. 146, 114-120 (1991) and Glimcher, L. H. and Kara, C. J. Ann. Rev. Immunol. 10, 13-49 (1992) and references therein. These agents include cytokines, antibodies to other cell surface molecules and phorbol esters. One agent which unpregulates MHC class I and class II molecules on a wide variety of cell types is the cytokine interferon-γ. Thus, for example, tumor cells modified to express B7-2 and/or B7-3 can be further modified to increase expression of MHC molecules by contact with interferon-γ.

Another agent which can be used to induce or increase expression of ah MHC molecule on a tumor cell surface is a nucleic acid encoding a transcription factor which upregulates transcription of MHC class I or class II genes. Such a nucleic acid can be transfected into the tumor cell to cause increased transcription of MHC genes, resulting in increased cell-surface levels of MHC proteins. MHC class I and class II genes are regulated by different transcription factors. However, the multiple MHC class I genes are regulated coordinately, as are the multiple MHC class II genes. Therefore, transfection of a tumor cell with a nucleic acid encoding a transcription factor which regulates MHC gene expression may increase expression of several different MHC molecules on the tumor cell surface. Several transcription factors which regulate the expression of MHC genes have been identified, cloned and characterized. For example, see Reith, W. et al., Genes Dev. 4, 1528-1540, (1990); Liou, H.-C., et al., Science 247, 1581-1584 (1988); Didier, D. K., et al., Proc. Natl. Acad. Sci. USA 85, 7322-7326 (1988).

III. Inhibition of Invariant Chain Expression in Tumor Cells

Another embodiment of the invention provides a tumor cell modified to express a T cell costimulatory molecule (e.g., B7-2 and/or B7-3) and in which expression of an MHC class II-associated protein, the invariant chain, is inhibited. Invariant chain expression is inhibited to promote association of endogenously-derived TAA peptides with MHC class II molecules to create an antigen-MHC complex. This complex can trigger an antigen-specific signal in T cells to induce activation of T cells in conjunction with a costimulatory signal. MHC class II molecules have been shown to be capable of presenting endogenously-derived peptides. Nuchtern, J. G., et al. Nature 343, 74-76 (1990); Weiss, S. and Bogen, B. Cell 767-776 (1991). However, in cells which naturally express MHC class II molecules, the α and β chain proteins are associated with the invariant chain (hereafter Ii) during intracellular transport of the proteins from the endoplasmic reticulum. It is believed that Ii functions in part by preventing the association of endogenously-derived peptides with MHC class II molecules. Elliott, W., et al. J. Immunol. 138, 2949-2952 (1987); Stockinger, B., et al. Cell 56, 683-689 (1989); Guagliardi, L., et al. Nature (London) 343, 133-139 (1990); Bakke, O., et al. Cell 63, 707-716 (1990); Lottreau, V., et al. Nature 348,600-605 (1990); Peters, J., et al. Nature 349, 669-676 (1991); Roche, P., et al. Nature 345, 615-618 (1990); Teyton, L., et al. Nature 348, 39-44 (1990). Since TAAs are synthesized endogenously in tumor cells, peptides derived from them are likely to be available intracellularly. Accordingly, inhibiting the expression of Ii in tumor cells which express Ii may increase the likelihood that TAA peptides will associate with MHC class II molecules. Consistent with this mechanism, it was shown that supertransfection of an MHC class II⁺. Ii⁻ tumor cell with the Ii gene prevented stimulation of tumor-specific immunity by the tumor cell. Clements, V. K., et al. J. Immunol. 149, 2391-2396 (1992).

Prior to modification, the expression of Ii in a tumor cell can be assessed by detecting the presence or absence of Ii mRNA by Northern blotting or by detecting the presence or absence of Ii protein by immunoprecipitation. A preferred approach for inhibiting expression of Ii is by introducing into the tumor cells a nucleic acid which is antisense to a coding or regulatory region of the Ii gene, which have been previously described. Koch, N., et al., EMBO J. 6, 1677-1683, (1987). For example, an oligonucleotide complementary to nucleotides near the translation initiation site of the Ii mRNA can be synthesized. One or more antisense oligonucleotides can be added to media containing tumor cells, typically at a concentration of oligonucleotides of 200 μg/ml. The antisense oligonucleotide is taken up by tumor cells and hybridizes to Ii mRNA to prevent translation. In another embodiment, a recombinant expression vector is used in which a nucleic acid encoding sequences of the Ii gene in an orientation such that mRNA which is antisense to a coding or regulatory region of the Ii gene is produced. Tumor cells transfected with this recombinant expression vector thus contain a continuous source of Ii antisense nucleic acid to prevent production of Ii protein. Alternatively, Ii expression in a tumor cell can be inhibited by treating the tumor cell with an agent which interferes with Ii expression. For example, a pharmaceutical agent which inhibits Ii gene expression, Ii mRNA translation or Ii protein stability or intracellular transport can be used.

IV. Types of Tumor Cells to be Modified

The tumor cells to be modified as described herein include tumor cells which can be transfected or treated by one or more of the approaches encompassed by the present invention to express B7-2 and/or B7-3, alone or in combination with B7. If necessary, the tumor cells can be further modified to express MHC molecules or an inhibitor of Ii expression. A tumor from which tumor cells are obtained can be one that has arisen spontaneously, e.g in a human subject, or may be experimentally derived or induced, e.g. in an animal subject. The tumor cells can be obtained, for example, from a solid tumor of an organ, such as a tumor of the lung, liver, breast, colon, bone etc. Malignancies of solid organs include carcinomas, sarcomas, melanomas and neuroblastomas. The tumor cells can also be obtained from a blood-borne (ie. dispersed) malignancy such as a lymphoma, a myeloma or a leukemia.

The tumor cells to be modified include those that express MHC molecules on their cell surface prior to transfection and those that express no or low levels of MHC class I and/or class II molecules. A minority of normal cell types express MHC class II molecules. It is therefore expected that many tumor cells will not express MHC class II molecules naturally. These tumors can be modified to express B7-2 and/or B7-3 and MHC class II molecules. Several types of tumors have been found to naturally express surface MHC class II molecules such as melanomas (van Duinen et al. Cancer Res. 48, 1019-1025, 1988), diffuse large cell lymphomas (O'Keane et al., Cancer 66, 1147-1153, 1990), squamous cell carcinomas of the head and neck (Mattijssen et al., Int. J. Cancer 6, 95-100, 1991) and colorectal carcinomas (Moller et al., Int. J. Cancer 6, 155-162, 1991). Tumor cells which naturally express class II molecules can be modified to express B7-2 and/or B7-3, and, in addition, other class II molecules which can increase the spectrum of TAA peptides which can be presented by the tumor cell. Most non-malignant cell types express MHC class I molecules. However, malignant transformation is often accompanied by downregulation of expression of MHC class I molecules on the surface of tumor cells. Csiba, A., et al., Brit. J Cancer 50, 699-709 (1984). Importantly, loss of expression of MHC class I antigens by tumor cells is associated with a greater aggressiveness and/or metastatic potential of the tumor cells. Schrier, P. I., et al. Nature 305, 771-775 (1983); Holden, C. A., et al. J. Am. Acad. Dermatol. 9., 867-871 (1983); Baniyash, M., et al. J. Immunol. 129, 1318-1323 (1982). Types of tumors in which MHC class I expression has been shown to be inhibited include melanomas, colorectal carcinomas and squamous cell carcinomas van Duinen et al., Cancer Res. 48, 1019-1025, (1988); Moller et al., Int. J. Cancer 6, 155-162, (1991); Csiba, A., et al., Brit. J Cancer 50, 699-709 (1984); Holden, C. A., et al. J. Am. Acad Dermatol. 9., 867-871 (1983). A tumor cell which fails to express class I molecules or which expresses only low levels of MHC class I molecules can be modified by one or more of the techniques described herein to induce or increase expression of MHC class I molecules on the tumor cell surface to enhance tumor cell immunogenicity.

V. Modification of Tumor Cells In Vivo

Another aspect of the invention provides methods for increasing the immunogenicity of a tumor cell by modification of the tumor cell in vivo to express B7-2 and/or B7-3 to trigger a costimulatory signal in T cells. In addition, tumor cells can be further modified in vivo to express MHC molecules to trigger a primary, antigen-specific, signal in T cells. Tumor cells can be modified in vivo by introducing a nucleic acid encoding B7-2 and/or B7-3 into the tumor cells in a form suitable for expression of the costimulatory molecule(s) on the surface of the tumor cells. Likewise, nucleic acids encoding MHC class I or class II molecules or an antisense sequence of the Ii gene can be introduced into tumor cells in vivo. In one embodiment, a recombinant expression vector is used to deliver nucleic acid encoding B7-2 and/or B7-3 to tumor cells in vivo as a form of gene therapy. Vectors useful for in vivo gene therapy have been previously described and include retroviral vectors, adenoviral vectors and adeno-associated viral vectors. See e.g. Rosenfeld, M. A., Cell 68, 143-155 (1992); Anderson, W. F., Science 226, 401-409 (1984); Friedman, T., Science 244, 1275-1281 (1989). Alternatively, nucleic acid can be delivered to tumor cells in vivo by direct injection of naked nucleic acid into tumor cells. See e.g. Acsadi, G., et al., Nature 332, 815-818 (1991). A delivery apparatus is commercially available (BioRad). Optionally, to be suitable for injection, the nucleic acid can be complexed with a carrier such as a liposome. Nucleic acid encoding an MHC class I molecule complexed with a liposome has been directly injected into tumors of melanoma patients. Hoffman, M., Science 256, 305-309 (1992).

Tumor cells can also be modified in vivo by use of an agent which induces or increases expression of B7-2 and/or B7-3 (and, if necessary, MHC molecules) as described herein. The agent may be administered systemically, e.g. by intravenous injection, or, preferably, locally to the tumor cells.

VI. The Effector Phase of the Anti-Tumor T Cell-Mediated Immune Response

The modified tumor cells of the invention are useful for stimulating an anti-tumor T cell-mediated immune response by triggering an antigen-specific signal and a costimulatory signal in tumor-specific T cells. Following this inductive, or afferent, phase of an immune response, effector populations of T cells are generated. These effector T cell populations can include both CD4+ T cells and CD8+ T cell. The effector populations are responsible for elimination of tumors cell, by, for example, cytolysis of the tumor cells. Once T cells are activated, expression of a costimulatory molecule is not required on a target cell for recognition of the target cell by effector T cells or for the effector functions of the T cells. Harding, F. A. and Allison, J. P. J. Exp. Med. 177, 1791-1796 (1993). Therefore, the anti-tumor T cell-mediated immune response induced by the modified tumor cells of the invention is effective against both the modified tumor cells and unmodified tumor cells which do not express a costimulatory molecule.

Additionally, the density and/or type of MHC molecules on the cell surface required for the afferent and efferent phases of a T cell-mediated immune response can differ. Fewer MHC molecules, or only certain types of MHC molecules (e.g. MHC class I but not MHC class II) may be needed on a tumor cell for recognition by effector T cells than is needed for the initial activation of T cells. Therefore, tumor cells which naturally express low amounts of MHC molecules but are modified to express increased amounts of MHC molecules can induce a T cell-mediated immune response which is effective against the unmodified tumor cells. Alternatively, tumor cells which naturally express MHC class I molecules but not, MHC class II molecules which are then modified to express MHC class II molecules can induce a T cell-mediated immune response which includes effector T cell populations which can eliminate the parental MHC class I+, class II− tumor cells.

VII. Therapeutic Compositions of Tumor Cells

Another aspect of the invention is a composition of modified tumor cells in a biologically compatible form suitable for pharmaceutical administration to a subject in vivo. This composition comprises an amount of modified tumor cells and a physiologically acceptable carrier. The amount of modified tumor cells is selected to be therapeutically effective. The term “biologically compatible form suitable for pharmaceutical administration in vivo” means that any toxic effects of the tumor cells are outweighed by the therapeutic effects of the tumor cells. A “physiologically acceptable carrier” is one which is biologically compatible with the subject. Examples of acceptable carriers include saline and aqueous buffer solutions. In all cases, the compositions must be sterile and must be fluid to the extent that easy syringability exists. The term “subject” is intended to include living organisms in which tumors can arise or be experimentally induced. Examples of subjects include humans, dogs, cats, mice, rats, and transgenic species thereof.

Administration of the therapeutic compositions of the present invention can be carried out using known procedures, at dosages and for periods of time effective to achieve the desired result. For example, a therapeutically effective dose of modified tumor cells may vary according to such factors as age, sex and weight of the individual, the type of tumor cell and degree of tumor burden, and the immunological competency of the subject. Dosage regimens may be adjusted to provide optimum therapeutic responses. For instance, a single dose of modified tumor cells may be administered or several doses may be administered over time. Administration may be by injection, including intravenous, intramuscular, intraperitoneal and subcutaneous injections.

VIII. Activation of Tumor-Specific T Lymphocytes In Vitro

Another approach to inducing or enhancing an anti-tumor T cell-mediated immune response by triggering a costimulatory signal in T cells is to obtain T lymphocytes from a tumor-bearing subject and activate them in vitro by stimulating them with tumor cells and a stimulatory form of B7-2 and/or B7-3. T cells can be obtained from a subject, for example, from peripheral blood. Peripheral blood can be further fractionated to remove red blood cells and enrich for or isolate T lymophocytes or T lymphocyte subpopulations. T cells can be activated in vitro by culturing the T cells with tumor cells obtained from the subject (e.g. from a biopsy or from peripheral blood in the case of blood-borne malignancies) together with a stimulatory form of B7-2 and/or B7-3 or, alternatively, by exposure to a modified tumor cell as described herein. The term “stimulatory form” means that the costimulatory molecule is capable of crosslinking its receptor on a T cell and triggering a costimulatory signal in T cells. The stimulatory form of the costimulatory molecule can be, for example, a soluble multivalent molecule or an immobilized form of the costimulatory molecule, for instance coupled to a solid support. Fragments, mutants or variants (e.g. fusion proteins) of B7-2 and/or B7-3 which retain the ability to trigger a costimulatory signal in T cells can also be used. In a preferred embodiment, a soluble extracellular portion of B7-2 and/or B7-3 is used to provide costimulation to the T cells. Following culturing of the T cells in vitro with tumor cells and B7-2 and/or B7-3, or a modified tumor cell, to activate tumor-specific T cells, the T cells can be administered to the subject, for example by intravenous injection.

IX. Therapeutic Uses of Modified Tumor Cells

The modified tumor cells of the present invention can be used to increase tumor immunogenicity, and therefore can be used therapeutically for inducing or enhancing T lymphocyte-mediated anti-tumor immunity in a subject with a tumor or at risk of developing a tumor. A method for treating a subject with a tumor involves obtaining tumor cells from the subject, modifying the tumor cells ex vivo to express a T cell costimulatory molecule, for example by transfecting them with an appropriate nucleic acid, and administering a therapeutically effective dose of the modified tumor cells to the subject. Appropriate nucleic acids to be introduced into a tumor cell include nucleic acids encoding B7-2 and/or B7-3, alone or together with nucleic acids encoding B7, MHC molecules (class I or class II) or Ii antisense sequences as described herein. Alternatively, after tumor cells are obtained from a subject, they can be modified ex vivo using an agent which induces or increases expression of B7-2 and/or B7-3 (and possibly also using agent(s) which induce or increase B7 or MHC molecules).

Tumor cells can be obtained from a subject by, for example, surgical removal of tumor cells, e.g. a biopsy of the tumor, or from a blood sample from the subject in cases of blood-borne malignancies. In the case of an experimentally induced tumor, the cells used to induce the tumor can be used, e.g. cells of a tumor cell line. Samples of solid tumors may be treated prior to modification to produce a single-cell suspension of tumor cells for maximal efficiency of transfection. Possible treatments include manual dispersion of cells or enzymatic digestion of connective tissue fibers, e.g. by collagenase.

Tumor cells can be transfected immediately after being obtained from the subject or can be cultured in vitro prior to transfection to allow for further characterization of the tumor cells (e.g. determination of the expression of cell surface molecules). The nucleic acids chosen for transfection can be determined following characterization of the proteins expressed by the tumor cell. For instance, expression of MHC proteins on the cell surface of the tumor cells and/or expression of the Ii protein in the tumor cell can be assessed. Tumors which express no, or limited amounts of or types of MHC molecules (class I or class II) can be transfected with nucleic acids encoding MHC proteins; tumors which express Ii protein can be transfected with Ii antisense sequences. If necessary, following transfection, tumor cells can be screened for introduction of the nucleic acid by using a selectable marker (e.g. drug resistance) which is introduced into the tumor cells together with the nucleic acid of interest.

Prior to administration to the subject, the modified tumor cells can be treated to render them incapable of further proliferation in the subject, thereby preventing any possible outgrowth of the modified tumor cells. Possible treatments include irradiation or mitomycin C treatment, which abrogate the proliferative capacity of the tumor cells while maintaining the ability of the tumor cells to trigger antigen-specific and costimulatory signals in T cells and thus to stimulate an immune response.

The modified tumor cells can be administered to the subject by injection of the tumor cells into the subject. The route of injection can be, for example, intravenous, intramuscular, intraperitoneal or subcutaneous. Administration of the modified tumor cells at the site of the original tumor may be beneficial for inducing local T cell-mediated immune-responses against the original tumor. Administration of the modified tumor cells in a disseminated manner, e.g. by intravenous injection, may provide systemic anti-tumor immunity and, furthermore, may protect against metastatic spread of tumor cells from the original site. The modified tumor cells can be administered to a subject prior to or in conjunction with other forms of therapy or can be administered after other treatments such as chemotherapy or surgical intervention.

Additionally, more than one type of modified tumor cell can be administered to a subject. For example, an effective T cell response may require exposure of the T cell to more than one type of costimulatory molecule. Furthermore, the temporal sequence of exposure of the T cell to different costimulatory mocules may be important for generating an effective response. For example, it is known that upon activation, a B cell expresses B7-2 early in its response (about 24 hours after stimulation). Subsequently, B7 and B7-3 are expressed by the B cell (about 48-72 hours after stimulation). Thus, a T cell may require exposure to B7-2 early in the induction of an immune response by exposure to B7 and/or B7-3 in the immune response. Accordingly, different types of modified tumor cells can be administered at different times to a subject to generate an effective immune response against the tumor cells. For example, tumor cells modified to express B7-2 can be administered to a subject. Following this administration, a tumor cell from the same tumor but modified to express B7-3 (alone or in conjunction with B7) can be administered to the subject.

Another method for treating a subject with a tumor is to modify tumor cells in vivo to express B7-2 and/or B7-3, alone or in conjunction with B7, MHC molecules and/or an inhibitor of Ii expression. This method can involve modifying tumor cells in vivo by providing nucleic acid encoding the protein(s) to be expressed using vectors and delivery methods effective for in vivo gene therapy as described in a previous section herein. Alternatively, one or more agents which induce or increase expression of B7-2 and/or B7-3, and possibly B7 or MHC molecules, can be administered to a subject with a tumor.

The modified tumor cells of the current invention may also be used in a method for preventing or treating metastatic spread of a tumor or preventing or treating recurrence of a tumor. As demonstrated in detail in one of the following examples, anti-tumor immunity induced by B7-expressing tumor cells is effective against subsequent challenge by tumor cells, regardless of whether the tumor cells of the re-exposure express B7 or not. Thus, administration of modified tumor cells or modification of tumor cells in vivo as described herein can provide tumor immunity against cells of the original, unmodified tumor as well as metastases of the original tumor or possible regrowth of the original tumor.

The current invention also provides a composition and a method for specifically inducing an anti-tumor response in CD4⁺ T cells. CD4⁺ T cells are activated by antigen in conjunction with MHC class II molecules. Association of peptidic fragments of TAAs with MHC class II molecules results in recognition of these antigenic peptides by CD4⁺ T cells. Providing a subject with tumor cells which have been modified to express MHC class II molecules along with B7-2 and/or B7-3, or modified in vivo to express MHC class II molecules along with B7-2 and/or B7-3, can be useful for directing tumor antigen presentation to the MHC class II pathway and thereby result in antigen recognition by and activation of CD4⁺ T cells specific for the tumor cells. As explained in detail in an example to follow, depletion of either CD4⁺ or CD8⁺ T cells in vivo, by administration of anti-CD4 or anti-CD8 antibodies, can be used to demonstrate that specific anti-tumor immunity is mediated by a particular (e.g. CD4⁺) T cell subpopulation.

As demonstrated in Example 2, subjects initially exposed to modified tumor cells develop an anti-tumor specific T cell response which is effective against subsequent exposure to unmodified tumor cells. Thus the subject develops anti-tumor specific immunity. The generalized use of modified tumor cells of the invention from one human subject as an immunogen to induce anti-tumor immunity in another human subject is prohibited by histocompatibility differences between unrelated humans. However, use of modified tumor cells from one individual to induce anti-tumor immunity in another individual to protect against possible future occurrence of a tumor may be useful in cases of familial malignancies. In this situation, the tumor-bearing donor of tumor cells to be modified is closely related to the (non-tumor bearing) recipient of the modified tumor cells and therefore the donor and recipient share MHC antigens. A strong hereditary component has been identified for certain types of malignancies, for example certain breast and colon cancers. In families with a known susceptibility to a particular malignancy and in which one individual presently has a tumor, tumor cells from that individual could be modified to express B7-2 and/or B7-3 and administered to susceptible, histocompatible family members to induce an anti-tumor response in the recipient against the type of tumor to which the family is susceptible. This anti-tumor response could provide protective immunity to subsequent development of a tumor in the immunized recipient.

X. Tumor-Specific T Cell Tolerance

In the case of an experimentally induced tumor, such as described in Examples 1 to 3, a subject (e.g. a mouse) can be exposed to the modified tumor cells of the invention before being challenged with unmodified tumor cells. Thus, the subject is initially exposed to TAA peptides on tumor cells together with B7-2 and/or B7-3, which activates TAA-specific T cells. The activated T cells are then effective against subsequent challenge with unmodified tumor cells. In the case of a spontaneously arising tumor, as is the case with human subjects, the subject's immune system will be exposed to unmodified tumor cells before exposure to the modified tumor cells of the invention. Thus the subject is initially exposed to TAA peptides on tumor cells in the absence of a costimulatory signal. This situation is likely to induce TAA-specific T cell tolerance in those T cells which are exposed to and are in contact with the unmodified tumor cells. Secondary exposure of the subject to modified tumor cells which can trigger a costimulatory signal may not be sufficient to overcome tolerance in TAA-specific T cells which were anergized by primary exposure to the tumor. Use of modified tumor cells to induce anti-tumor immunity in a subject already exposed to unmodified tumor cells may therefore be most effective in early diagnosed patients with small tumor burdens, for instance a small localized tumor which has not metastasized. In this situation, the tumor cells are confined to a limited area of the body and thus only a portion of the T cell repertoire may be exposed to tumor antigens and become anergized. Administration of modified tumor cells in a systemic manner, for instance after surgical removal of the localized tumor and modification of isolated tumor cells, may expose non-anergized T cells to tumor antigens together with B7-2 and/or B7-3, thereby inducing an anti-tumor response in the non-anergized T cells. The anti-tumor response may be effective against possible regrowth of the tumor or against micrometastases of the original tumor which may not have been detected. To overcome widespread peripheral T cell tolerance to tumor cells in a subject, additional signals, such as a cytokine, may need to be provided to the subject together with the modified tumor cells. A cytokine which functions as a T cell growth factor, such as IL-2, could be provided to the subject together with the modified tumor cells. IL-2 has been shown to be capable of restoring the alloantigen-specific responses of previously anergized T cells in an in vitro system when exogenous IL-2 is added at the time of secondary alloantigenic stimulation. Tan, P., et al. J. Exp. Med. 177, 165-173 (1993).

Another approach to generating an anti-tumor T cell response in a subject despite tolerance of the subject's T cells to the tumor is to stimulate an anti-tumor response in T cells from another subject who has not been exposed to the tumor (referred to as a naive donor) and transfer the stimulated T cells from the naive donor back into the tumor-bearing subject so that the transferred T cells can mount an immune response against the tumor cells. An anti-tumor response is induced in the T cells from the naive donor by stimulating the T cells in vitro with the modified tumor cells of the invention. Such an adoptive transfer approach is generally prohibited in outbred populations because of histocompatibity differences between the transferred T cells and the tumor-bearing recipient. However, advances in allogeneic bone marrow transplantation can be applied to this situation to allow for acceptance by the recipient of the adoptively transferred cells and prevention of graft versus host disease. First, a tumor-bearing subject (referred to as the host) is prepared for and receives an allogeneic bone marrow transplant from a naive donor by a known procedure. Preparation of the host involves whole body irradiation, which destroys the host's immune system, including T cells tolerized to the tumor, as well as the tumor cells themselves. Bone marrow transplantation is accompanied by treatment(s) to prevent craft versus host disease such as depletion of mature T cells from the bone marrow graft, treatment of the host with immunosuppressive drugs or treatment of the host with an agent, such as CTLA4Ig, to induce donor T cell tolerance to host tissues. Next, to provide anti-tumor specific T cells to the host which can respond against residual tumor cells in the host or regrowth or metastases of the original tumor in the host, T cells from the naive donor are stimulated in vitro with tumor cells from the host which have been modified, as described herein, to express B7-2 and/or B7-3. Thus, the donor T cells are initially exposed to tumor cells together with a costimulatory signal and therefore are activated to respond to the tumor cells. These activated anti-tumor specific T cells are then transferred to the host where they are reactive against unmodified tumor cells. Since the host has been reconstituted with the donor's immune system, the host will not reject the transferred T cells and, additionally, the treatment of the host to prevent graft versus host disease will prevent reactivity of the transferred T cells with normal host tissues.

This invention is further illustrated by the following examples which should not be construed as limiting. The contents of all references and published patents and patent applications cited throughout the application are hereby incorporated by reference.

In Examples 1-3, mouse sarcoma cells were modified to express the T cell costimulatory molecule B7. The following methodology was used in Examples 1 to 3.

Methods and Materials

A. Cells

SaI tumor cells were maintained as described (Ostrand-Rosenberg, S., et al., J. Immunol. 144, 4068-4071 (1990)).

B. Antibodies

The monoclonal antibody (mAb) 10-3.6, specific for I-A^{k} (Oi, V., et al. Curr. Top. Microbiol. Immunol. 81, 115-120 (1978)), was prepared and used as described. Ostrand-Rosenberg, S., et al., J. Immunol. 144: 4068-4071 (1990). The B7-specific mAb 1G10 is a rat IgG2a mAb and was used as described (Nabavi, N., et al. Nature 360, 266-268 (1992)). mAbs specific for CD4⁺ [GK1.5 (Wilde, D. B., et al. J. Immunol. 131, 2178-2183 (1983))] and CD8⁺ [2.43 (Sarmiento, M., et al. J. Immunol. 125, 2665-2672 (1980))] were used as ascites fluid.

C. Transfections

Mouse SaI sarcoma cells were transfected as described in Ostrand-Rosenberg, S., et al., J. Immunol. 144, 4068-4071 (1990). SaI cells (2×10⁶) were transfected by the calcium phosphate method (Wigler et al., Proc. Natl. Acad. Sci. USA, 76, 1373 (1979)). SaI cells were transfected with wild-type Aα^{k} and Aβ^{k} MHC class II cDNAs (Ostrand-Rosenberg, S., et al., J. Immunol. 144, 4068-4071 (1990)), Aα^{k} and Aβ^{k} cDNAs truncated for their C-terminal 12 and 10 amino acids, respectively (Nabavi, N., et al. J. Immunol. 142, 1444-1447 (1989)), and/or B7 gene (Freeman. G. J. et al. J. Exp. Med. 174, 625-631 (1991)). For transfection, the murine B7 cDNA was subcloned into the eukaryotic expression vector dCDNAI (Invitrogen, San Diego, Calif.). Class II transfectants were cotransfected with pSV2neo plasmid and selected for resistance to G418 (400 μg/ml). B7 transfectants were cotransfected with pSV2hph plasmid and selected for hygromycin-resistance (400 μg/ml). All transfectants were cloned twice by limiting dilution, except SaI/B7 transfectants, which were uncloned, and maintained in drug. Double transfectants were maintained in G418 plus hygromycin. The numbers after each transfectant are the clone designation.

D. Immunofluorescence

Indirect immunofluorescence was performed as described (Ostrand-Rosenberg. S., et al., J. Immunol. 144, 4068-4071 (1990)), and samples were analyzed on an Epics C flow cytometer.

E. Tumor Challenges

For primary tumor challenges, autologous A/J mice were challenged intraperitoneally (i.p.) with the indicated number of tumor cells. Inoculated mice were checked three times per week for tumor growth. Mean survival times of mice dying from their tumor ranged from 13 to 28 days after inoculation. Mice were considered to have died from their tumor if they contained a large volume of ascites fluid and tumor cells (≧5 ml) at the time of death. Mice were considered tumor-resistant if they were tumor-free for at least 60 days after tumor challenge (range, 60-120 days). Tumor cells were monitored by indirect immunofluorescence for I-A^{k} and B7 expression prior to tumor-cell inoculation. For the experiments of Table 2, autologous A/J mice were immunized i.p. with a single inoculum of the indicated number of live tumor cells and challenged i.p. with the indicated number of wild-type SaI cells 42 days after immunization. Mice were evaluated for tumor resistance or susceptibility using the same criteria as for primary tumor challenge.

F. In vivo T cell Depletions

A/J mice were depleted of CD4⁺ or CD8⁺ T cells by i.p. inoculation with 100 μl of ascites fluid of mAb GK1.5 (CD4⁺ specific; Wilde, D. B., et al., J. Immunol. 131, 2178-2183 (1983)) or mAb 2.43 (CD8⁺ specific; Sarmiento, M., et al., J. Immunol. 125, 2665-2672 (1980)) on days-6,-3, and -1 prior to tumor challenge, and every third day after tumor challenge as described (Ghobrial, M., et al. Clin. Immunol. Immunopathol. 52, 486-506 (1989)) until the mice died or day 28, whichever came first. Presence or absence of tumor was assessed up to day 28. Previous studies have established that A/J mice with large tumors at day 28 after injection will progress to death. This time point was, therefore, chosen to assess tumor susceptibility for the in vivo depletion experiments. One mouse per group was sacrificed on day 28, and its spleen was assayed by immunofluorescence to ascertain depletion of the relevant T cell population.

### EXAMPLE 1

### Coexpression of B7 Restores Tumor Immunogenicity

A mouse sarcoma cell line SaI was used in each of the examples. The mouse SaI sarcoma is an ascites-adapted class I⁺ class II⁻ tumor of A/J (H-2K^{k}A^{k}D^{d}) mice. The wild-type tumor is lethal in autologous A/J mice when administered i.p. It has previously been shown that SaI cells transfected with, and expressing, syngeneic MHC class II genes (Aα^{k} and Aβ^{k} genes; SaI/A^{k} cells) are immunologically rejected by the autologous host, and immunization with live SaI/A^{k} cells protects mice against subsequent challenges with wild-type class II⁻ SaI cells (Ostrand-Rosenberg, S., et al., J. Immunol. 144, 4068-4071 (1990)). Adoptive transfer (Cole, G., et al. Cell. Immunol. 134, 480-490 (1991)) and lymphocyte depletion studies (E. Lamousse-Smith and S. O.-R., unpublished data) demonstrate that SaI and SaI/A^{k} rejection is dependent on CD4⁺ lymphocytes. SaI cells expressing class II molecules with truncated cytoplasmic domains (SaI/A^{k}tr cells), however, are as lethal as wild-type class II⁻ SaI cells, suggesting that the cytoplasmic region of the class II heterodimer is required to induce protective immunity (Ostrand-Rosenberg, S., et al. J. Immunol. 147, 2419-2422 (1991)).

Up-regulation of the B7 activation molecule on APCs is triggered by intracellular signals transmitted by the cytoplasmic domain of the class II heterodimer, after presentation of antigen to CD4⁺ T helper cells (Nabavi, N., et al., Nature 360, 266-268 (1992)). Inasmuch as B7 expression is normally up-regulated in vivo on SaI cells expressing full-length class II molecules (S. B. and S. O.-R., unpublished data), it may be that SaI/A^{k}tr cells do not stimulate protective immunity because they do not transmit a costimulatory signal.

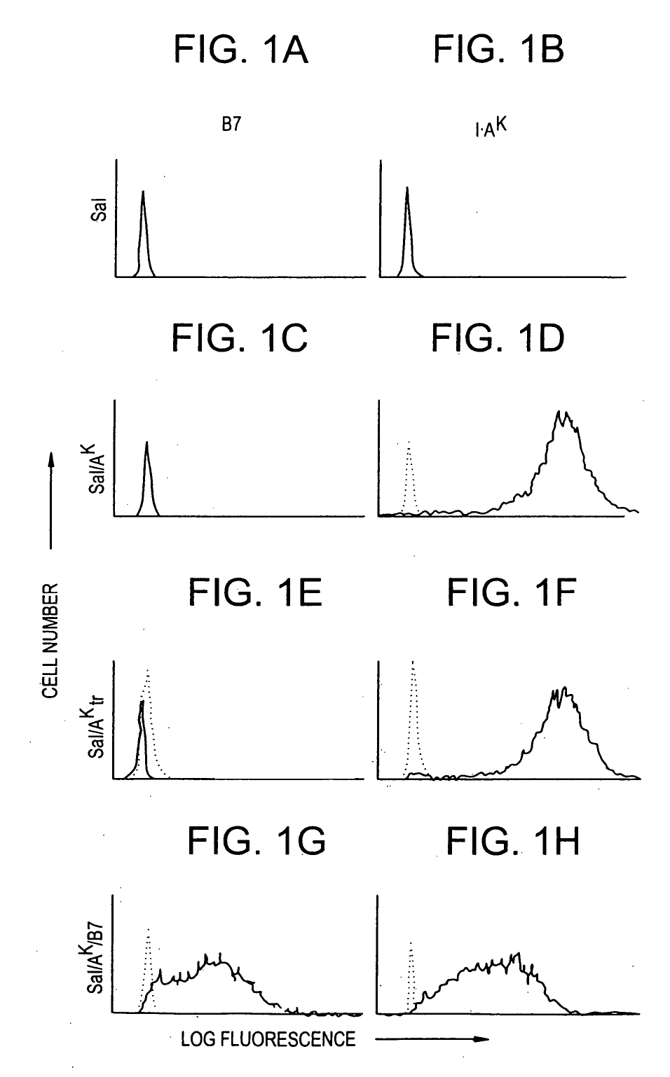

To test whether B7 expression can compensate for the absence of the class II cytoplasmic domain, SaI/A^{k}tr cells were supertransfected with a plasmid containing a cDNA encoding murine B7 under the control of the cytomegalovirus promoter and screened for I-A^{k} and B7 expression by indirect immunofluorescrence. Wild-type SaI cells do not express either I-A^{k} or B7 (FIGS. 1a and b), whereas SaI cells transfected with Aα^{k} and Aβ^{k} genes (SaI/A^{k} cells) express I-A^{k} (FIGS. 1d and f) and do not express B7 (FIGS. 1c and e). SaI cells transfected with truncated class II genes plus the B7 gene (SaI/A^{k}tr/B7 cells) express I-A^{k} and B7 molecules (FIGS. 1g and h). All cells express uniform levels of MHC class I molecules (K^{k} and D^{d}) comparable to the level of I-A^{k} in FIG. 1h.

Antigen-presenting activity of the transfectants was tested by determining their immunogenicity and lethality in autologous A/J mice. As shown in Table 1, wild-type SaI cells administered i.p. at doses as low as 10⁴ cells are lethal in 88-100% of mice inoculated within 13-28 days after challenge, whereas 100 times as many SaI/A^{k} cells are uniformly rejected. Challenges with similar quantities of SaI/A^{k}tr cells are also lethal; however, SaI/A^{k}tr cells that coexpress B7 (SaI/A^{k}tr/B7 clone-1 and clone-3) are uniformly rejected. A/J mice challenged with SaI/A^{k}tr cells transfected with the B7 construct, but not expressing detectable amounts of B7 antigen (SaI/A^{k}tr/hph cells), are as lethal as SaI/A^{k}tr cells, demonstrating that reversal of the malignant phenotype in SaI/A^{k}tr/B7 cells is due to expression of B7. SaI cells transfected with the B7 gene and not coexpressing truncated class II molecules (SaI/B7 cells, uncloned) are also as lethal as wild-type SaI cells, indicating the B7 expression without truncated class II molecules does not stimulate immunity. To ascertain that rejection of SaI/A^{k} and SaI/A^{k}tr/B7 cells is immunologically mediated, sublethally irradiated (900 rads; 1 rad=0.01 Gy) A/J mice were challenged i.p. with these cells. In all cases, irradiated mice died from the tumor. Thus, immunogenicity and host rejection of the MHC class II⁺ tumor cells are dependent on an intact class II molecule and that coexpression of B7 can bypass the requirement of the class II intracellular domain.

TABLE 1
Tumorigenicity of B7 and MHC class II-transfected SaI tumor cells

| | | | Tumor | |
|---|---|---|---|---|
| | Expression | | dose, | Mice dead/mice |
| Challenge tumor | I-A^{k} | B7 | no. of cells | tested, no./no. |
| SaI | — | — | 1 × 10⁶ | 9/10 |
| | — | — | 1 × 10⁵ | 8/10 |
| | — | — | 1 × 10⁴ | 7/8 |
| SaI/A^{k} 19.6.4 | A^{k} | — | 1 × 10⁶ | 0/12 |
| | A^{k} | — | 5 × 10⁵ | 0/5 |
| | A^{k} | — | 1 × 10⁵ | 0/5 |
| SaI/A^{k}tr 6.11.8 | A^{k}tr | — | 1 × 10⁶ | 12/12 |
| | A^{k}tr | — | 5 × 10⁵ | 5/5 |
| | A^{k}tr | — | 1 × 10⁵ | 5/10 |
| SaI/A^{k}tr/B7-Clone 1 | A^{k}tr | B7 | 1 × 10⁶ | 0/4 |
| SaI/A^{k}tr/B7-Clone 3 | A^{k}tr | B7 | 1 × 10⁶ | 0/5 |
| | A^{k}tr | B7 | 4 × 10⁵ | 0/5 |
| | A^{k}tr | B7 | 1 × 10⁵ | 0/5 |
| SaI/A^{k}tr/hph | A^{k}tr | — | 1 × 10⁶ | 5/5 |
| SaI/B7 | — | B7 | 1 × 10⁶ | 5/5 |

### EXAMPLE 2

### Immunization with B7-Transfected Sarcoma Cells Protects Against Later Challenges of Wild-Type B7-Sarcoma

Activation of at least some T cells is thought to be dependent on coexpression of B7. However, once the T cells are activated, B7 expression is not required on the target T cell for recognition by effector T cells. The ability of three SaI/A^{k}tr/B7 clones (B7-clone 3, B7-clone 1, and B7-2B5.F2) to immunize A/J mice against subsequent challenges of wild-type class II⁻ B7⁻ SaI cells (Table 2) was determined. A/J mice were immunized with live SaI/A^{k}tr/B7 transfectants and 42 days later challenged with wild-type SaI tumor cells. Ninety-seven percent of mice immunized with SaI/A^{k}tr/B7 transfectants were immune to ≧10⁶ wild-type B7⁻ class II⁻ SaI cells, an immunity that is comparable to that induced by immunization with SaI cells expressing full-length class II molecules. SaI/A^{k}tr/B7 cells, therefore, stimulate a potent response with long-term immunological memory against high-dose challenges of malignant tumor cells. B7 expression is, therefore, critical for the stimulation of SaI-specific effector cells; however, its expression is not needed on the tumor targets once the appropriate effector T cell populations have been generated.

TABLE 2
Autologous A/J mice immunized with SaI/A^{k}tr/B7 cells
are immune to challenges of wild-type SaI tumor

| | | SaI | |
|---|---|---|---|
| | | challenge | |
| | | dose | Mice dead/ |
| | No. of | no. of | mice tested |
| Immunization | immunizing cells | cells | no./no. |
| None | — | 1 × 10⁶ | 5/5 |
| SaI/A^{k} 19.6.4 | 1 × 10⁵ or 10⁶ | 1 × 10⁶ | 0/5 |
| | 1 × 10⁶ | 6 × 10⁶ | 0/5 |
| SaI/A^{k}tr/B7-clone 3 | 1 × 10⁶ | 6 × 10⁶ | 0/5 |
| | 1 × 10⁶ | 1 × 10⁶ | 0/5 |
| | 4 × 10⁵ | 1 × 10⁶ | 0/5 |
| | 1 × 10⁵ | 5 × 10⁶ | 0/5 |
| SaI/A^{k}tr/B7-clone 1 | 5 × 10⁵ | 3 × 10⁶ | 0/3 |
| | 2 × 10⁵ | 1 × 10⁶ | 0/2 |
| | 5 × 10⁴ | 5 × 10⁶ | 0/3 |
| SaI/A^{k}tr/B7-2B5.E2 | 1 × 10⁵ | 2 × 10⁶ | 0/2 |
| | 5 × 10⁴ | 2 × 10⁶ | 1/7 |

### EXAMPLE 3

### Immunization with B7-Transfected Tumor Cells Stimulates Tumor-Specific CD4^{±} Lymphocytes

To ascertain that B7 is functioning through a T cell pathway in tumor rejection, we have in vivo-depleted A/J mice for CD4⁺ or CD8⁺ T cells and challenged them i.p with SaI/A^{k} or SaIA^{k}tr/B7 cells. As shown in Table 3, in vivo depletion of CD4⁺ T cells results in host susceptibility to both SaI/A^{k} and SaI/A^{k}tr/B7 tumors, indicating that CD4⁺ T cells are critical for tumor rejection, whereas depletion of CD8⁺ T cells does not affect SaI/A^{k}tr/B7 tumor rejection. Although immunofluorescence analysis of splenocytes of CD8⁺-depleted mice demonstrates the absence of CD8⁺ T cells, it is possible that the depleted mice contain small quantitites of CD8⁺ cells that are below our level of detection. These data therefore demonstrate that CD4⁺ T cells are required for tumor rejection but do not eliminate a possible corequirement for CD8⁺ T cells.

TABLE 3
Tumor susceptibility of A/J mice in vivo-depleted for
CD4⁺ or CD8⁺ T cells

| | | No. mice with tumor/ |
|---|---|---|
| Tumor challenge | Host T cell depletion | total no. mice challenged |
| SaI/A^{k} | CD4⁺ | 3/5 |
| SaI/A^{k}tr/B7-clone 3 | CD4⁺ | 5/5 |
| | CD8⁺ | 0/5 |

Previous adoptive transfer experiments (Cole, G., et al. Cell. Immunol. 134, 480-490 (1991)) have demonstrated that both CD4⁺ and CD8⁺ T cells are required for rejection of class II wild-type SaI cells. Inasmuch as rejection of SaI/A^{k} and SaI/A^{k}tr/B7 cells appears to require only CD4⁺ T cells, it is likely that immunization with class II⁺ transfectants stimulates both CD4⁺ and CD8⁺ effector T cells; however, only the CD8⁺ effectors are required for rejection of class I⁺ II⁻ tumor targets. Costimulation by B7, therefore, enhances immunity by stimulating tumor-specific CD4⁺ helper and cytotoxic lymphocytes.

### EXAMPLE 4

### Determination of the Effect of Modified Tumor Cells in Subjects Previously Exposed to Unmodified Tumor Cells

In the previous examples, mice were immunized with modified tumor cells to which they had not been previously exposed. In the case of treating a subject with a pre-existing tumor, the subject will be exposed to unmodified tumor cells for a period of time before exposure to modified tumor cells, and therefore the subject may become tolerized to the unmodified tumor cells.

To determine whether the modified tumor cells of the invention which express B7-2 and/or B7-3 are effective in overcoming tolerance and inducing an anti-tumor T cell response in a subject, mice are inoculated with increasing amounts of wild-type SaI tumor cells which have been irradiated with 10,000 rads. Doses of tumor cells in the range of 1×10⁴ to 1×10⁶ cells can be inoculated. Tumor cells irradiated in this way survive for up to two months in the recipient mice, sufficient time for tolerance to the tumor cells to be induced in the mice. After two months exposure to the wild-type tumor cells, mice are injected simultaneously with wild-type tumor cells into the flank of one hind leg and with tumor cells modified to express B7-2 and/or B7-3 into the flank of the opposite hind leg. As a control, mice are injected with wild-type tumor cells into both flanks. Tumor cell doses in the range of 1×10⁴ to 1×10⁶ cells are used for challenges. Tumor growth is assessed by measuring the size of a tumor which grows at the site of injection. The ability of tumor cells modified to express B7-2 and/or B7-3 to induce anti-tumor immunity, and therefore overcome any possible tolerance to the tumor cells in the mice, is determined by the ability of the modified tumor cells injected into one flank to prevent growth of wild-type tumor cells in the opposite flank, as compared to when wild-type tumor cells are injected into both flanks.

Alternatively, the ability of tumor cells modified to express B7-2 and/or B7-3 to overcome potential tolerance to unmodified tumor cells is assessed by an adoptive transfer experiment. A mouse is injected intraperitoneally with a low dose, e.g. 1×10⁴ cells, of wild-type SaI cells and the tumor cells are allowed to grow for three weeks, at which time the mouse is sacrificed and spleen cells from the mouse are harvested. These spleen cells are injected intraperitoneally into a recipient, syngeneic mouse which has been lethally irradiated to destroy its endogenous immune system. The adoptively transferred spleen cells reconstitute the recipient mouse with an immune system which has been previously exposed to wild-type tumor cells. Following spleen cell transfer, the recipient mouse is then challenged with wild-type tumor cells injected into the flank of one hind leg and with tumor cells modified to express B7-2 and/or B7-3 injected into the flank of the opposite hind leg. Tumor cell doses in the range of 1×10⁴ to 1×10⁶ cells are used for challenges. The ability of the modified tumor cells to induce anti-tumor immunity is determined by the ability of the modified tumor cells injected into one flank to prevent the growth of wild-type tumor cells injected into the opposite flank.

### EXAMPLE 5

### Regression of Implanted Tumor Cells Transfected to Express B7-2

In this example, untransfected or B7-2 transfected J558 plasmacytoma cells were used in tumor regression studies to examine the effect of expression of B7-2 on the surface of tumor cells on the growth of the tumor cells when transplanted into animals.

J558 plasmacytoma-cells (obtained from the American Type Culture Collection, Rockville, Md.; # TIB 6) were transfected with an expression vector containing cDNA encoding either mouse B7-2 (pAWNE03) or B7-1 (PNRDSH or pAWNE03) and a neomycin-resistance gene. Stable transfectants were selected based upon their neomycin resistance and cell surface expression of B7-2 or B7-1 on the tumor cells was confirmed by FACS analysis using either an anti-B7-2 or anti-B7-1 antibody.

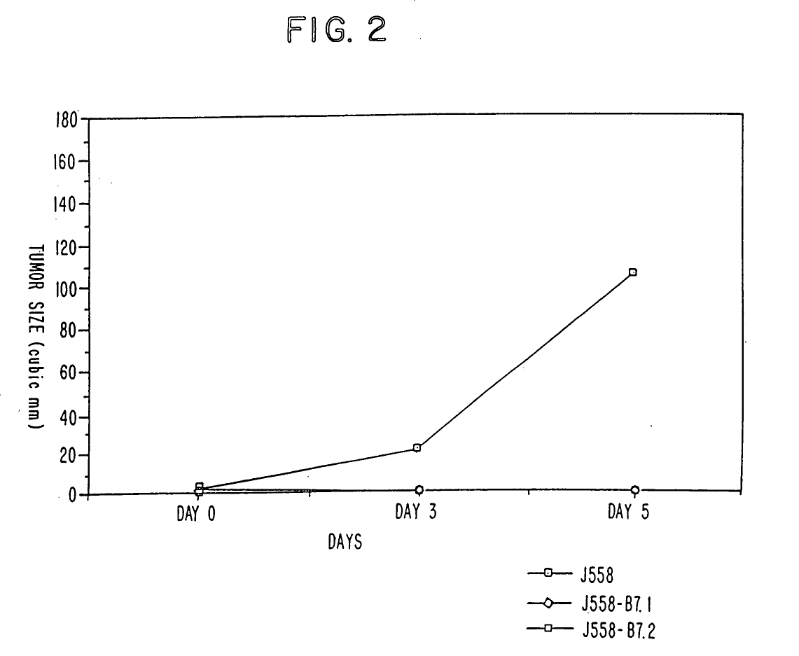

Syngeneic Balb/c mice, in groups of 5-10 mice/set, were used in experiments designed to determine whether cell-surface expression of B7-2 on tumor cells would result in regression of the implanted tumor cells. Untransfected and transfected J558 cells were cultured in vitro, collected, washed and resuspended in Hank's buffered salt solution (GIBCO, Grand Island, N.Y.) at a concentration of 10⁸ cells/ml. A patch of skin on the right flank of each mouse was removed of hair with a depilatory and, 24 hours later, 5×10⁶ tumor cells/mouse were implanted intradermally or subdermally. Measurements of tumor volume (by linear measurements in three perpendicular directions) were made every two to three days using calipers and a ruler. A typical experiment lasted 18-21 days, after which time the tumor size exceeded 10% of the body mass of mice transplanted with untransfected, control J558 cells. As shown in FIG. 2, J558 cells transfected to express B7-2 on their surface were rejected by the mice. No tumor growth was observed even after three weeks. Similar results were observed with J558 cells transfected to express B7-1 on their surface. In contrast, the untransfected (wild-type) J558 cells produced massive tumors in as little as 12 days, requiring the animal to be euthanized. This example demonstrates that cell-surface expression of B7-2 on tumor cells, such as by transfection of the tumor cells with a B7-2 cDNA, induces an anti-tumor response in naive animals that is sufficient to cause rejection of the tumor cells.

Equivalents

Those skilled in the art will recognize, or be able to ascertain using no more than routine experimentation, many equivalents to the specific embodiments of the invention described herein. Such equivalents are intended to be encompassed by the following claims.

1. A method for treating a mammalian subject having a solid tumor ex vivo, comprising direct injection of a nucleic acid molecule encoding a B7-2 molecule in a form suitable for expression of the B7-2 molecule, into cells of the tumor, wherein the B7-2 molecule has the ability to costimulate a T cell and the ability to bind a CD28 or CTLA4 ligand, such that the growth of the tumor is inhibited.

2. A method for modifying cells of a solid tumor ex vivo to express a B7-2 molecule comprising, direct injection of a nucleic acid molecule encoding a B7-2 molecule in a form suitable for expression of the B7-2 molecule, into the tumor cells, wherein the B7-2 molecule has the ability to costimulate a T cell and the ability to bind a CD28 or CTLA4 ligand, such that B7-2 is expressed by the tumor cells.

3. A method of increasing the immunogenecity of a cells of a solid tumor ex vivo comprising, direct injection of a nucleic acid molecule encoding a B7-2 molecule in a form suitable for expression of the B7-2 molecule, into the tumor cells, wherein the B7-2 molecule has the ability to costimulate a T cell and the ability to bind a CD28 or CTLA4 ligand, such that B7-2 is expressed by the tumor cells, to thereby increase the immunogenicity of the tumor cells.

4. The method of any of claims 1-3, wherein the nucleic acid molecule encoding a B7-2 molecule comprises the nucleic sequence shown in SEQ ID NO:1.

5. The method of any of claims 1-3, wherein B7-2 comprises the amino acid sequence shown in SEQ ID NO:2.

6. The method of any of claims 1-3, wherein the nucleic acid molecule encoding B7-2 is in a viral vector.

7. The method of claim 6, wherein the viral vector is selected from the group consisting of a retroviral vector, an adenoviral vector, and an adeno-associated viral vector.

8. The method of any of claims 1-3, wherein the nucleic acid molecule encoding B7-2 is a plasmid expression vector.

9. The method of any of claims 1-3, wherein the tumor cells are further transfected with at least one nucleic acid molecule encoding a B7-3 protein.

10. The method of any of claims 1-3, wherein the tumor cells are further injected with at least one nucleic acid molecule encoding at least one MHC class II α chain protein and at least one MHC class II β chain protein in a form suitable for expression of the MHC class II α chain protein(s) and the MHC class II β chain protein(s).

11. The method of any of claims 1-3, wherein the tumor cells are further injected with at least one nucleic acid molecule encoding at least one MHC class I α chain protein in a form suitable for expression of the MHC class I protein(s).

12. The method of any of claims 1-3, wherein the tumor cells are further injected with a nucleic acid molecule encoding a β-2 microglobulin protein in a form suitable for expression of the β-2 microglobulin protein.

13. The method of any of claims 1-3, wherein expression of the MHC class II invariant chain is inhibited in the tumor cells by transfection of the tumor cells with a nucleic acid molecule which is antisense to a regulatory or a coding region of the invariant chain gene.

14. The method of any of claims 1-3 wherein the solid tumor is selected from a group consisting of a carcinoma, sarcoma, melanoma and neuroblastoma.


SEQUENCE LISTING (8 sequences)

SEQ ID NO: 1 - DNA (cDNA), 1120 bp; single-stranded, linear
  CDS 107..1093
cacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccaaaaaaaa

SEQ ID NO: 2 - protein, 329 aa
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 3 - DNA (cDNA), 1151 bp; double-stranded, linear
  CDS 99..1028
ggagcaagcagacgcgtaagagtggctcctgtaggcagcacggacttgaacaaccagactcctgtagacgtgttccagaacttacggaagcacccacgatggaccccagatgcaccatgggcttggcaatccttatctttgtgacagtcttgctgatctcagatgctgtttccgtggagacgcaagcttatttcaatgggactgcatatctgccgtgcccatttacaaaggctcaaaacataagcctgagtgagctggtagtattttggcaggaccagcaaaagttggttctgtacgagcactatttgggcacagagaaacttgatagtgtgaatgccaagtacctgggccgcacgagctttgacaggaacaactggactctacgacttcacaatgttcagatcaaggacatgggctcgtatgattgttttatacaaaaaaagccacccacaggatcaattatcctccaacagacattaacagaactgtcagtgatcgccaacttcagtgaacctgaaataaaactggctcagaatgtaacaggaaattctggcataaatttgacctgcacgtctaagcaaggtcacccgaaacctaagaagatgtattttctgataactaattcaactaatgagtatggtgataacatgcagatatcacaagataatgtcacagaactgttcagtatctccaacagcctctctctttcattcccggatggtgtgtggcatatgaccgttgtgtgtgttctggaaacggagtcaatgaagatttcctccaaacctctcaatttcactcaagagtttccatctcctcaaacgtattggaaggagattacagcttcagttactgtggccctcctccttgtgatgctgctcatcattgtatgtcacaagaagccgaatcagcctagcaggcccagcaacacagcctctaagttagagcgggatagtaacgctgacagagagactatcaacctgaaggaacttgaaccccaaattgcttcagcaaaaccaaatgcagagtgaaggcagtgagagcctgaggaaagagttaaaaattgctttgcctgaaataagaagtgcagagtttctcagaattcaaaaatgttctcagctgattggaattctacagttgaataattaaagaac

SEQ ID NO: 4 - protein, 309 aa
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAE

SEQ ID NO: 5 - DNA (cDNA to mRNA), 1491 bp, Homo sapien; double-stranded, linear
  original source tissue type: lymphoid
  original source cell type: B cell
  original source cell line: Raji
ccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctgaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtagcctccgtcatctcttctgggatacatggatcgtggggatcatgaggcattcttcccttaacaaatttaagctgttttacccactacctcaccttcttaaaaacctctttcagattaagctgaacagttacaagatggctggcatccctctcctttctccccatatgcaatttgcttaatgtaacctcttcttttgccatgtttccattctgccatcttgaattgtcttgtcagccaattcattatctattaaacactaatttgag

SEQ ID NO: 6 - protein, 288 aa
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 7 - DNA (cDNA to mRNA), 1716 bp, Mus musculus; double-stranded, linear
  original source developmental stage: germ line
  original source tissue type: lymphoid
  original source cell type: B lymphocyte
  original source cell line: 70Z and A20
gagttttatacctcaatagactcttactagtttctctttttcaggttgtgaaactcaaccttcaaagacactctgttccatttctgtggactaataggatcatctttagcatctgccgggtggatgccatccaggcttctttttctacatctctgtttctcgatttttgtgagcctaggaggtgcctaagctccattggctctagattcctggctttccccatcatgttctccaaagcatctgaagctatggcttgcaattgtcagttgatgcaggatacaccactcctcaagtttccatgtccaaggctcaatcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctttagttcttctctgtccatgtgggatacatggtattatgtggctcatgaggtacaatctttctttcagcaccgtgctagctgatctttcggacaacttgacacaagatagagttaactgggaagagaaagccttgaatgaggatttctttccatcaggaagctacgggcaagtttgctgggcctttgattgcttgatgactgaagtggaaaggctgagcccactgtgggtggtgctagccctgggcaggggcaggtgaccctgggtggtataagaaaaagagctgtcactaaaaggagaggtgcctagtcttactgcaacttgatatgtcatgtttggttggtgtctgtgggaggcctgcccttttctgaagagaagtggtgggagagtggatggggtgggggcagaggaaaagtgggggagagggcctgggaggagaggagggagggggacggggtgggggtggggaaaactatggttgggatgtaaaaacggataataatataaatattaaataaaaagagagtattgagcaaaaaaaaaaaaaaaaaa

SEQ ID NO: 8 - protein, 306 aa
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYGVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL